(19) United States
(12) Patent Application Publication
(10) Pub. No.: US 2026/0335223 A1
(43) Pub. Date: Oct. 29, 2026

(54) Topical Copolymer Formulations for Hydrophobic Drug Delivery

(71) Applicant: 1376683 B.C. LTD, Vancouver (CA)

(72) Inventors: John K. Jackson, West Vancouver (CA); Dana M. Lambert, North Vancouver (CA)

(21) Appl. No.: 19/143,182

(22) PCT Filed: Feb. 7, 2024
(86) PCT No.: PCT/CA2024/050150

§ 371 Date: Jun. 25, 2025

Related U.S. Application Data
(60) Provisional application No. 63/445,650, filed on Feb. 14, 2023.

(51) Int. Cl. A61K 9/00; A61K 31/00; A61K 47/10; A61K 47/34; A61P 19/02; A61P 29/02

(52) U.S. Cl. CPC A61K 9/0014; A61K 31/658; A61K 47/10; A61K 47/34; A61P 19/02; A61P 29/02

(57) ABSTRACT

The invention provides topical drug delivery formulations in which selected diblock copolymers (DBCs) solubilize a range of hydrophobic drugs in topical pharmaceutical dosage forms.

### FIELD

The invention is in the field of pharmaceutical formulations, comprising hydrophobic active agents delivered topically in block copolymer formulations.

### BACKGROUND

The skin acts as a protective barrier, both limiting entry of foreign substances into the body and preventing loss of endogenous material. Human skin possesses physical, chemical and biological properties that restrict the types of agents that can permeate through its barrier and enter the body. The efficacy of the skin as a barrier is reflected in the fact that many drugs do not readily penetrate the skin. This is largely due to the stratum corneum, the outermost skin layer. The stratum corneum consists of dead cells known as corneocytes embedded in an intercellular matrix of lipids (see Elias, 1983). The lipids are comprised of ceramides (~50%), cholesterol (~25%), cholesterol sulfate (~5%), fatty acids (10-15%), and a small amount of cholesterol esters, with little or no phospholipids (Wertz and Downing, 1996). There are at least nine types of ceramides, and they are known to play a principal role in the skin's barrier function. Generally, this barrier acts to physically block molecules as well as provide an unfavorable hydrophobic/hydrophilic balance for drug penetration. For a drug to permeate the skin via passive diffusion, it must have specific properties, such as molecular weight, molecular dimensions, pKa and Log P which are well suited to the skin's anatomy and physiology. The molecular weight cut off for molecules by size exclusion is generally recognized as approximately 500 daltons. These requirements have limited the commercial availability of dermal and transdermal drug products.

Numerous methods have been tried to overcome the stratum corneum barrier to drug transport including microneedles, electrical, ultrasonic, eutectic drug mixtures, carrier solvents and complex dermal formulation design. Physical methods like microneedles, ultrasound and electrical stimulation require complex equipment. Formulation design using solvents like DMSO or Transcutol® may work to some degree but are often impractical due to the potential toxicity effects of local solvent uptake into the body.

It has been previously reported that certain diblock copolymer micelles are capable of solubilizing drugs such as paclitaxel, and such micelles can carry up to 20% drug by weight (Zhang, 1996; Zhang, 1997; Letchford and Burt, 2012). Unfortunately, the ability of micelles to solubilize drugs is not universal and many significantly hydrophobic drugs (e.g., camptothecin with a water solubility of 1 μg/mL) do not form drug-loaded micelles. In general, there are significant challenges associated with formulating drugs that are characterized for pharmaceutical purposes as “practically insoluble”, i.e. having a solubility of ≤100 μg/ml (on a scale of descending solubilities: “very soluble” being >1000 mg/ml soluble, “freely soluble” being 100-1000 mg/ml soluble, “soluble” being 33-100 mg/ml soluble, “sparingly soluble” being 10-33 mg/ml soluble, “slightly soluble” being 1-10 mg/ml soluble, and “very slightly soluble” being 0.1-1 mg/ml soluble).

Cannabinoids like CBD, THC, CBG, CBC, CBN and acidic forms thereof are all hydrophobic compounds with water solubilities close to 1 μg/mL, which have been formulated for topical use (Stella, 2021; Bruni, 2018). For topical and transdermal formulations, simple solvent methods have been used to increase the solubility of these agents (e.g. ethanol, propylene glycol, olive oil; Stella, 2021; Bruni, 2018), as well as other approaches (Momekova et al., 2020; Paudel, 2010).

### SUMMARY

Topical drug delivery formulations are provided in which certain diblock copolymers (DBCs) solubilize a range of cannabinoids and other hydrophobic drugs with high efficiency, such as drugs that are practically insoluble. As exemplified herein, the use of DBCs allows formulation of these hydrophobic drugs into water-based transdermal gels and solid film dosage forms, and the DBC gel formulations may be used to greatly enhance the skin permeation of the drugs and the DBC solid film formulations may be used to greatly enhance drug release. The present DBC transdermal gel formulations result in demonstrably improved therapeutic outcomes compared to those achieved with control gel formulations lacking DBC.

Diblock copolymers for use in the present formulations may be composed of a hydrophilic chain (such as polyethylene glycol (PEG), methoxy-PEG (MePEG)) in combination with a hydrophobic chain such as a polylactide (PLLA), poly(lactic-co-glycolic acid) (PLGA), polycaprolactone (PCL), polydodecyl glycol, polypropylene glycol (PPG) or polymethyl methacrylate. In select embodiments, formulations are provided with a 60:40 (weight ratio) of methoxypoly(ethylene glycol) (MePEG): poly-DL-lactide (PLLA) and a molecular weight (MW) of ~3333 Daltons (referred to herein as “DBC₃₃₃₃”). In alternative embodiments, DBCs made from PCL instead of PLLA also demonstrably work as solubilizing agents and facilitate hydrophobic drug delivery. For example, a DBC ratio of 57 wt % MePEG to 43 wt % PCL with a total molecular weight of 1320 greatly enhanced delivery of cannabinoids into skin tissue. A similar polymer with 40% MePEG to 60% PCL and a total molecular weight of 1890 also facilitated hydrophobic drug delivery. A similar polymer with 63% MePEG to 37% PCL and a total molecular weight of 7900 greatly increased hydrophobic drug delivery into skin.

In keeping with the exemplified embodiments, formulations are provided comprising low molecular weight diblock copolymers (less than approximately 10,000 Da) with select hydrophilic to hydrophobic ratios may be used to solubilize cannabinoids and other hydrophobic agents in water-based transdermal preparations for enhanced drug uptake in the skin to achieve improved therapeutic outcomes. In select embodiments, the hydrophilic to hydrophobic ratios in the present formulations may for example be greater than 35:65, or greater than 40:60, or from 35:65 to 65:35, or from 40:60 to 60:40. In select embodiments of the present formulations, the DBC may be present in an amount of from 0.5-20% w/w or 2-6% w/w; and/or each hydrophobic drug may for example be present in an amount of from 0.1-15% w/w or 1-12% w/w; and/or, the pharmaceutical carrier may be present in an amount of from 55-99% w/w or 75-97% w/w.

In select embodiments, a topical pharmaceutical formulation is provided, comprising or consisting essentially of: a diblock copolymer (DBC) having a molecular weight of ≤10 kDa, comprised of a hydrophilic block polymer copolymerized with a hydrophobic block polymer, wherein the hydrophilic block polymer and the hydrophobic block polymer are present in the DBC in a weight ratio of from 35:65 to 65:35; and, a hydrophobic active agent entrained with the DBC as a dispersion in a topical pharmaceutical carrier, wherein the hydrophobic active agent has a solubility in water of ≤100 μg/ml (or wherein the hydrophobic active agent is practically insoluble).

### BRIEF DESCRIPTION OF THE DRAWINGS

FIG. 1 is a bar graph illustrating the effect of diblock copolymer DCP₃₃₃₃ on THCA and THC skin permeation.

FIG. 2 is a bar graph illustrating the effect of diblock DCP₃₃₃₃ copolymer on cannabidiol skin permeation.

FIG. 3 is a bar graph illustrating the effect of diblock copolymer DCP₃₃₃₃ on curcumin skin permeation.

FIG. 4 is a bar graph illustrating the effect of diblock copolymer DCP₃₃₃₃ on finasteride skin permeation.

FIG. 5 is a bar graph illustrating the effect of diblock copolymer DCP₃₃₃₃ on docetaxel skin permeation.

FIG. 6 is a bar graph illustrating the effect of diblock copolymer DCP₃₃₃₃ on cannabinol skin permeation.

FIG. 7 is a bar graph illustrating the effect of diblock copolymer PCL₅ on cannabidiol skin permeation.

FIG. 8 is a bar graph illustrating the effect of diblock copolymer PCL₂₅ on capsaicin skin permeation.

FIG. 9 is a line graph illustrating the release of curcumin from polyvinyl alcohol (PVA) films.

### DETAILED DESCRIPTION

The present pharmaceutical compositions may be adapted for topical administration, to be applied directly to a part of the body, using a pharmaceutically acceptable carrier to prepare a solution, film, dispersion, emulsion, ointment, cream or gel base. The base, for example, may comprise one or more of the following: petrolatum, lanolin, polyethylene glycols, polyvinyl alcohols, bee wax, mineral oil, glycerin, diluents such as water and alcohol and/or phenoxyethanol, and emulsifiers and stabilizers. Thickening and/or gelling agents may be present in a pharmaceutical composition for topical administration. Topical medicaments disclosed herein may for example be applied to any body surface, internally or externally. If intended for transdermal administration, the composition may include a transdermal patch or iontophoresis device. Hydrophobic active agents may be entrained with the DBC, for example as a dispersion, for example in a topical pharmaceutical carrier, in the sense that the DBC formulation delivers or carries along the active agent as an entrained component of the formulation. Some hydrophobic active agents may for example be solubilized to varying degrees, some may be otherwise entrained.

In exemplary embodiments, alternative formulations may include a variety of biologically active molecules derived from hemp or cannabis plants, for example various cannabinoids, such as cannabigerol (CBG), cannabichromene (CBC), tetrahydrocannabivarin (THCV), tetrahydrocannabinolic acid (THCA), tetrahydrocannabinol (THC), cannabidiol (CBD), and cannabinol (CBN). Biologically active molecules may for example include terpenes (isoprenoids), such as: α-pinene, ocimene, caryophyllene (β-caryophyllene), camphene, camphor, eucatyptol, humulene (α-humulene), myrcene, gamma-terpinene, cis-nerolidol, carene, terpinolene, terpineol, trans-nerolidol, cymene (p-cymene), linalool, phellandrene, guaiol, limonene, iso-pulegol, cary-oxide, a-terpinene, geraniol, valencene, fenchol, borneol (isoborneol), phytol, sabinene, menthol, cedrene, nerolidol, isopulegol, geranyl acetate, pulegone, bisabolol. Alternative biologically active agents for inclusion in the present formulations include curcumin, finasteride, docetaxel, indomethacin, diclofenac, ibuprofen, naproxen, celecoxib, diflusinal, etodolac, flurbiprofen, capsaicin, psilocin and resveratrol.

### EXAMPLES

### Example 1: DBC Enhanced Solubility

This Example illustrates that select DBCs enhance the aqueous solubility of hydrophobic drugs (as shown in Tables 1-8). In these studies the cannabinoid and diblock copolymer are dissolved in ethanol and dried to a film. Warm water is added and solubility is assessed by an optically clear solution or via HPLC or UV/Vis spectrophotometry. Tables 1-6 illustrate the use of the BDC designated herein as DBC₃₃₃₃. Tables 7-12 illustrate the use of polymers referred to as PCL₅, PCL₁₀, PCL₂₅ and PCL₆₁, which respectively are: 57:43 MePEG:PCL, MW 1320 Da “PCL₅”; 40:60 MePEG:PCL, MW 1890 Da “PCL₁₀”; 63:37 MePEG:PCL MW 7900 Da “PCL₂₅”; and 30:70 MePEG:PCL, MW 12000 Da “PCL₆₁”.

Specific methodologies for aqueous solubility studies were as follows. Drug solutions having a concentration of 10 mg/mL were prepared by weighing drug and dissolving in an appropriate amount of ethanol in glass vials. Control drug samples (no diblock copolymer) were prepared by measuring 1 mL of room temperature distilled water in a glass vial and adding an appropriate volume of 10 mg/mL ethanol drug solution, followed by gentle mixing.

Drug samples with diblock copolymer (60:40 MePEG: PLLA, MW 3333 Da “DBC₃₃₃₃”; 57:43 MePEG:PCL, MW 1320 Da “PCL₅”; 40:60 MePEG:PCL, MW 1890 Da “PCL₁₀”; 30:70 MePEG:PCL, MW 12000 Da “PCL₆₁”) were prepared by weighing diblock copolymer into glass vials then adding 800 microlitres ethanol followed by an appropriate volume of 10 mg/mL ethanol drug solution. The diblock copolymer was dissolved in the ethanol solution via gentle mixing and the solvent was then evaporated via warm oven heating at approximately 50 °C. Once completely dry, 1 ml distilled water was added to the vials followed by gentle mixing. All vials were capped and left at room temperature for 12 hours, at which time the vials were photographed for evidence of precipitation.

Aqueous solubility studies involving diblock copolymer PCL₂₅ (63:37 MePEG:PCL MW 7900 Da) were carried out as follows. 1 mg of drug (either paclitaxel, capsaicin or curcumin) was added to glass vials, designated as either “control” or “diblock copolymer”. Diblock copolymer was omitted from control vials and 100 mg PCL₂₅ was added to diblock copolymer vials. The contents of all vials were then dissolved in 300 microlitres of acetonitrile. The contents were dried at 60 °C for two hours and then 1 mL of warm water (40 °C) was added with vortexing for five minutes. To determine the limit of drug solubility, the contents of each vial were then placed in a microcentrifuge tube and centrifuged at 14000×g for 3 minutes to sediment any undissolved solids. The supernatant was diluted 50% in acetonitrile and the drug was quantitated by HPLC for paclitaxel and capsaicin or by UV/VIS spectrometry (420 nm) for curcumin after 100 fold dilution in acetonitrile for this drug. HPLC conditions were detection at 228 nm for both drugs and a mobile phase of 60:40 (paclitaxel) or 50:50 (capsaicin) acetonitrile:water flowing at 1 mL/min. Calibration standard for all drugs were run in acetonitrile.

TABLE 1
Enhancement of aqueous solubility of THCA
with diblock copolymer DBC₃₃₃₃ 20 mg/mL

| | | | | | | THCA (μg/mL) with Diblock | | | | |
|---|---|---|---|---|---|---|---|---|---|---|
| THCA (μg/mL) Control | | | | | | Copolymer 20 mg/mL | | | | |
| 1 | 10 | 100 | 250 | 500 | 1000 | 250 | 500 | 1000 | 2000 | 3000 |
| Soluble | Insol. | Insol. | Insol. | Insol. | Insol. | Soluble | Soluble | Soluble | Partially | Insol. |
| | | | | | | | | | Soluble | |

TABLE 2
Enhancement of aqueous solubility of THC
with diblock copolymer DBC₃₃₃₃ 20 mg/mL

| | | | | | | THC (μg/mL) with Diblock | | | | |
|---|---|---|---|---|---|---|---|---|---|---|
| THC (μg/mL) Control | | | | | | Copolymer 20 mg/mL | | | | |
| 1 | 10 | 100 | 250 | 500 | 1000 | 250 | 500 | 1000 | 2000 | 3000 |
| Insol. | Insol. | Insol. | Insol. | Insol. | Insol. | Soluble | Soluble | Partially | Insol. | Insol. |
| | | | | | | | | Soluble | | |

TABLE 3
Enhancement of aqueous solubility of THC
with diblock copolymer DBC₃₃₃₃ 50 mg/mL

| | | | THC (μg/mL) with Diblock | | | | |
|---|---|---|---|---|---|---|---|
| THC (μg/mL) Control | | | Copolymer 50 mg/mL | | | | |
| 500 | 1000 | 2000 | 500 | 1000 | 2000 | 3000 | 4000 |
| Insol. | Insol. | Insol. | Soluble | Soluble | Soluble | Partially | Insol. |
| | | | | | | Soluble | |

TABLE 4
Enhanced aqueous solubility of CBD with
diblock copolymer DBC₃₃₃₃ 50 mg/mL

| | | | CBD (μg/mL) with Diblock | | | | |
|---|---|---|---|---|---|---|---|
| CBD (μg/mL) Control | | | Copolymer 50 mg/mL | | | | |
| 300 | 600 | 1000 | 300 | 600 | 1000 | 2000 | 3000 |
| Insol. | Insol. | Insol. | Soluble | Soluble | Soluble | Partially | Insol. |
| | | | | | | Soluble | |

TABLE 5
Enhanced aqueous solubility of CBG with
diblock copolymer DBC₃₃₃₃ 50 mg/mL

| CBG (μg/mL) | CBG (μg/mL) with Diblock | | | | |
|---|---|---|---|---|---|
| Control | Copolymer 50 mg/mL | | | | |
| 300 | 300 | 600 | 1000 | 2000 | 3000 |
| Insol. | Soluble | Soluble | Soluble | Partially | Insol. |
| | | | | Soluble | |

TABLE 6
Enhancement of aqueous solubility of finasteride
with diblock copolymer DBC₃₃₃₃ 50 mg/mL

| | | | Finasteride (μg/mL) with | | | | |
|---|---|---|---|---|---|---|---|
| Finasteride (μg/mL) Control | | | Diblock Copolymer 50 mg/mL | | | | |
| 300 | 600 | 1000 | 300 | 600 | 1000 | 2000 | 3000 |
| Insol. | Insol. | Insol. | Soluble | Partially | Insol. | Insol. | Insol. |
| | | | | Soluble | | | |

TABLE 7
Enhancement of aqueous solubility of THC with
PCL₅ and PCL₁₀ but not PCL₆₁ diblock copolymers

| | THC in diblock | | | | | | | | |
|---|---|---|---|---|---|---|---|---|---|
| | THC mg/ml | | | | | | | | |
| | 0.5 | 1 | 1 | 2 | 3 | 4 | 0.5 | 0.5 | 0.5 |
| | Diblock mg/ml | | | | | | | | |
| | | | Pcl 5 | Pcl 5 | Pcl 5 | Pcl 5 | Pcl 10 | Pcl5 + Pcl10 | Pcl 61 |
| | 0 | 0 | 50 | 50 | 50 | 50 | 22 | 22 + 2 | 22 |
| Solubility | Insol. | Insol. | soluble | soluble | soluble | Soluble | soluble | soluble | Insol. |

TABLE 8
Enhancement of aqueous solubility of THCA with
PCL₅ and PCL₁₀ but not PCL₆₁ diblock copolymers

| | THCA in diblock | | | | | | | | |
|---|---|---|---|---|---|---|---|---|---|
| | THCA mg/ml | | | | | | | | |
| | 0.5 | 1 | 1 | 2 | 3 | 4 | 0.5 | 0.5 | 0.5 |
| | Diblock mg/ml | | | | | | | | |
| | | | Pcl 5 | Pcl 5 | Pcl 5 | Pcl 5 | Pcl 10 | Pcl5 + Pcl10 | Pcl 61 |
| | 0 | 0 | 50 | 50 | 50 | 50 | 22 | 22 + 2 | 22 |
| Solubility | Insol. | Insol. | soluble | soluble | soluble | Soluble | soluble | soluble | Insol. |

TABLE 9
Enhancement of aqueous solubility of CBD with
PCL₅ and PCL₁₀ but not PCL₆₁ diblock copolymers

| | CBD in diblock | | | | | | | | |
|---|---|---|---|---|---|---|---|---|---|
| | CBD mg/ml | | | | | | | | |
| | 0.5 | 1 | 1 | 2 | 3 | 4 | 0.5 | 0.5 | 0.5 |
| | Diblock mg/ml | | | | | | | | |
| | | | Pcl 5 | Pcl 5 | Pcl 5 | Pcl 5 | Pcl 10 | Pcl5 + Pcl10 | Pcl 61 |
| | 0 | 0 | 50 | 50 | 50 | 50 | 22 | 22 + 2 | 22 |
| Solubility | Insol. | Insol. | soluble | soluble | Partially | Insol. | soluble | soluble | Insol. |
| | | | | | soluble | | | | |

TABLE 10
Enhancement of aqueous solubility of paclitaxel with
diblock copolymer PCL₂₅ 100 mg/mL

| Paclitaxel | Paclitaxel (μg/mL) with |
|---|---|
| (μg/mL) | Diblock Copolymer |
| Control | 100 mg/mL |
| 2 | 488 |
| Limit of solubility | Limit of solubility |

TABLE 11
Enhancement of aqueous solubility of capsaicin with
diblock copolymer PCL₂₅ 100 mg/mL

| Capsaicin | Capsaicin (μg/mL) with |
|---|---|
| (μg/mL) | Diblock Copolymer |
| Control | 100 mg/mL |
| 10 | 506 |
| Limit of solubility | Limit of solubility |

TABLE 12
Enhancement of aqueous solubility of curcumin with
diblock copolymer PCL₂₅ 100 mg/mL

| | Curcumin (μg/mL) with |
|---|---|
| Curcumin (μg/mL) | Diblock Copolymer |
| Control | 100 mg/mL |
| <1 | 1000 |
| Limit of solubility | Limit of solubility |

### Example 2: DBC Facilitated Gellation and Enhanced Skin Permeation

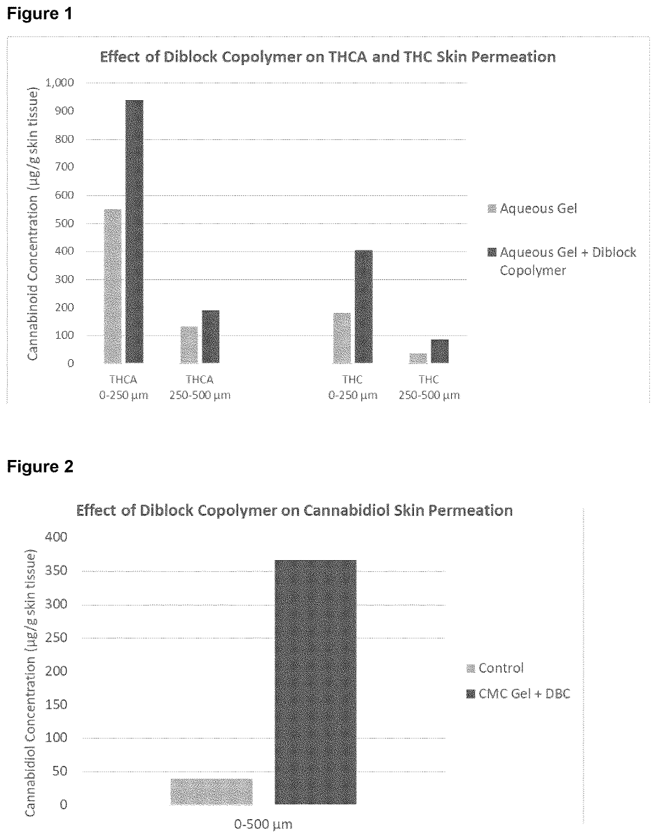

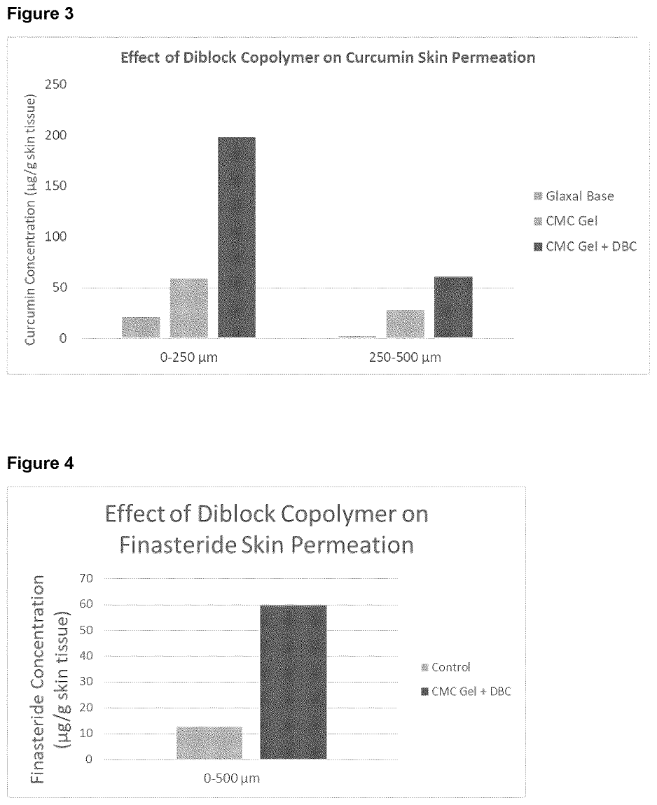

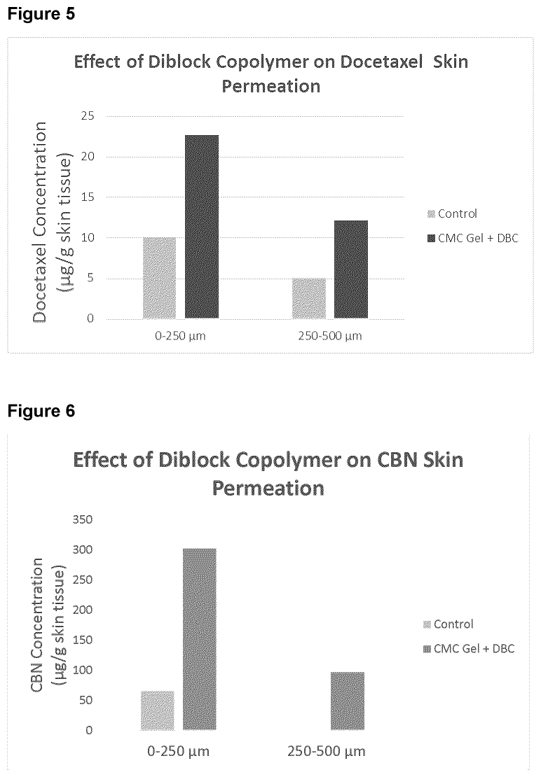

This Example illustrates that DBC₃₃₃₃ allowed for CBD, CBN, finasteride and docetaxel to be prepared within aqueous carboxymethyl cellulose (CMC) gels for topical use. Without the use of DBC₃₃₃₃, it was not possible to prepare homogenous CMC gel preparations containing these drugs due to solubility limitations (attempted gels contained solid particles and precipitated drug). Furthermore, DBC enhanced skin permeation of topically applied THCA, THC, CBD, CBN, curcumin, finasteride and docetaxel formulations (FIGS. 1-6).

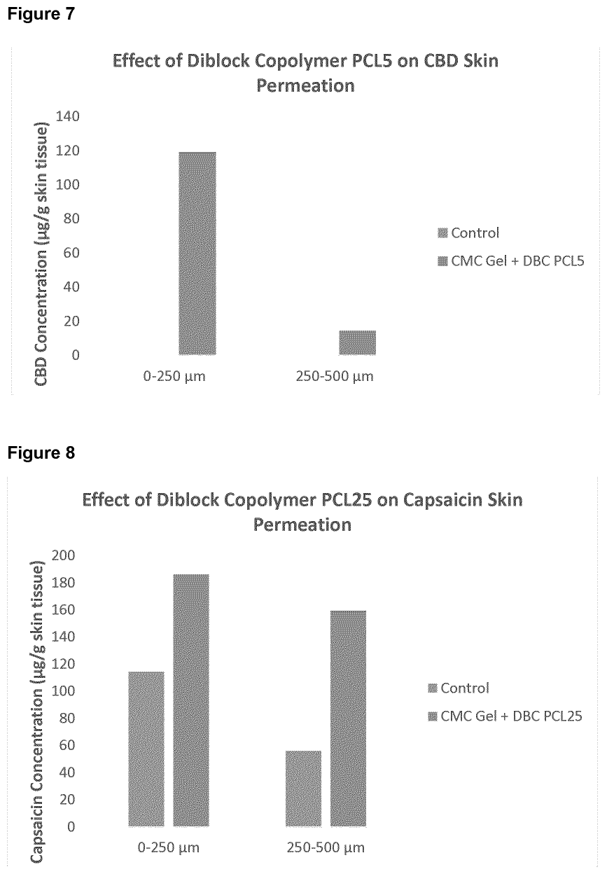

Similarly, PCL₅ diblock copolymer allowed for CBD to be prepared within an aqueous carboxymethyl cellulose (CMC) gel for topical use. Without the use of PCL₅ diblock copolymer, it was not possible to prepare homogenous CMC gel preparations containing CBD (attempted gels contained solid particles and precipitated drug) due to solubility limitations. Furthermore, PCL₅ diblock copolymer enhanced skin permeation of CBD from the topically applied CMC gel, compared to a control formula having identical CBD content prepared with Glaxal® base (FIG. 7).

Similarly, PCL₂₅ diblock copolymer allowed for capsaicin to be prepared within an aqueous carboxymethyl cellulose (CMC) gel for topical use. Without the use of PCL₂₅ diblock copolymer, it was not possible to prepare homogenous CMC gel preparations containing capsaicin (attempted gels contained solid particles and precipitated drug) due to solubility limitations. Furthermore, PCL₂₅ diblock copolymer enhanced skin permeation of capsaicin from the topically applied CMC gel, compared to a control formula having identical capsaicin content prepared with Glaxal® base (FIG. 8).

To collect the data illustrated in this Example, fresh pig skin is placed on a Franz diffusion cell and the formulation of interest is applied to the outer skin surface. After three hours the cell is dismantled and the exposed circular area excised. The top 0-250 μm deep section and lower 250-500 μm section of tissue are dissected out and weighed and the drug extracted in solvent. The amount of drug in each section is then quantitated and expressed at mass of drug/gram of tissue. Specific methodologies were as follows.

For cannabinoid aqueous gel preparation for skin permeation study and therapeutic efficacy studies, a water-based gel comprised of water, phenoxyethanol, ethylhexyl glycerin, acrylates/C10-30 alkyl acrylate crosspolymer, sodium hydroxide and allantoin, was used as the gel base. Gel base and diblock copolymer were weighed, mixed together and heated using a microwave for 4 seconds. A homogenous gel containing diblock copolymer was achieved via levigation of the mixture. Gel base without diblock copolymer was weighed and used for the control formula. Commercial cannabinoid-containing extracts containing known quantities of cannabinoid were weighed and dissolved in a pre-weighed amount of cacao butter over 45 °C heat. The cacao butter cannabinoid mixture was then weighed and incorporated into the gel base, with or without diblock copolymer, via levigation to produce the final gel samples. Cannabinoid content of the final gel samples was analyzed using HPLC. Gel had an opaque, light yellow, homogenous appearance, a smooth, homogenous texture and high viscosity. Gel samples were stored in air-tight containers at 4 °C until the time of use.

For carboxymethyl cellulose (CMC) gel and Glaxal® base preparation for skin permeation study of cannabidiol (CBD), curcumin, capsaicin and finasteride, CMC gel was made using medium viscosity CMC as a 2% w/v aqueous solution following overnight stirring. For CMC preparations not containing DBC, the drug was dissolved first in ethanol and blended directly into the CMC gel such that the final amount of ethanol did not exceed 0.2% v/v. For CMC preparations containing DBC, DBC was weighed into a glass vial and a small volume of the drug of interest in acetonitrile:methanol (50:50) was added to dissolve all. The solution was then dried for 2 hours in a warm oven (50 °C). In all cases the ratio of DBC:drug was 95:5. A known weight of CMC gel was then added to the warmed vial containing the dried DBC/drug mixture and vortexed to disperse and dissolve all drug. Glaxal® base was used as received. Glaxal® base containing drug was prepared by first weighing drug and dissolving in a known volume of ethanol or methanol. The ethanol or methanol drug solution was then added to a weighed amount of Glaxal® base followed by levigation of the mixture. For CBD, capsaicin, docetaxel and finasteride, it was not possible to prepare CMC gels without the use of DBC, due to drug insolubility. Therefore Glaxal® base preparations containing these drugs were employed as DBC-free comparison groups (control) for skin permeation studies.

For carboxymethyl cellulose (CMC) gel and Glaxal® base preparation for skin permeation study of docetaxel, 2% CMC gel was prepared as described above. Docetaxel (DTX) 6 mg was weighed into 190 μL of acetonitrile and 10 μL of 3H radiolabeled DTX was added. Then 66 μL (i.e., 2 mg of drug) was pipetted into a vial with 40 mg of DBC to dissolve all. The contents were dried down and 1 mL of warm 2% CMC gel was added with vortexing to dissolve all DBC and drug as a micellar solution. For Glaxal® paste 66 μL of the drug solution was dried down to approximately 20 μL and 1 gram of Glaxal® base added to disperse the drug.

Fresh pig skin was obtained from a butcher and defatted to 5 mm thickness and shaved. 2 cm² pieces were cut with scissors and placed on Franz diffusion cells filled with PBS and clamped. 100 mg of each formulation was weighed and placed on top of the tissue and capped. The cells were placed in a 37 °C humid incubator for three hours and then dismantled. The tissue was washed three times with PBS and the centre drug-loaded circle of tissue was cut out with scissors and snap frozen. Tissues were sectioned by cryosectioning into 50 μm cuts pooled into groups of five.

For cannabinoid experiments the same experiments were performed using tissues clamped between two cut syringes so the base was a syringe with plunger full of pbs and the top retaining cell was the cut stump of a syringe (i.e., containing the flanges that are held between fingers). These parts were then clamped over the exposed skin tissue as for other drug experiments. These tissues were then sectioned with a scalpel until a total thickness of tissue of 250 μm was taken from the top (measured using a micrometer) and then a 250-500 μm section below. All tissue sections were weighed.

300 microlitres of a 50:50 acetonitrile: methanol drug extraction solvent was added and the tubes capped and left overnight. Drug content of the extraction solvent was then measured using UV/VIS absorbance (curcumin at 420 nm) or HPLC for cannabinoids, capsaicin and finasteride or liquid scintillation counting for docetaxel. Data shown represent the mean of n=3 experimental replicates.

### Example 3: Enhanced Curcumin Release from PVA Film

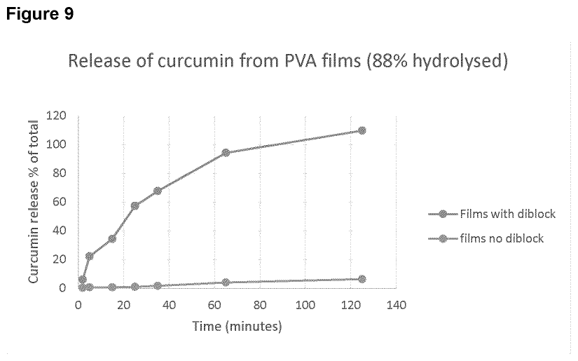

This Example illustrates the effect of DBC₃₃₃₃ on curcumin release from a polyvinyl alcohol (PVA) Film. PVA films formulated with no diblock copolymer dissolved very slowly at room temperature and released only low amounts of curcumin so that even by 2 hours only 6% of the drug had released. However, when diblock DBC₃₃₃₃ was added at 20% w/w to PVA, the release rate was much faster with all drug released by 2 hours as seen in FIG. 9. As an overall observation, the yellow colour in the diblock containing dish was markedly stronger compared to the no diblock film.

Data in this Example was obtained using the following methods. The 88% hydrolyzed version of PVA was used with a molecular weight of between 100000 and 150000 daltons. PVA was dissolved in water at 10% w/v by heating to 90 °C for one hour and then cooled. Separately, 40 mg of diblock copolymer DBC₃₃₃₃ was weighed into a glass vial and 100 μL of curcumin solution (10 mg/mL in ethanol) was added to 2 mL of the 10% PVA solution with mixing to give the non diblock casting solutions. Alternatively, 100 μL of the curcumin solution was added to 40 mg of diblock copolymer to dissolve it and then this was mixed with 2 mL of PVA solution. These give PVA films containing 20% diblock with approximately 0.5% curcumin (1 mg curcumin per film). The films were cast in small petri dishes and air dried at 37 °C for 4 hours.

This observational solubilization experiment added 2 mL of water to the film at t=0 and then at 5, 10, 25 mins the liquid was removed into a test tube for observation.

A second set of the same films were prepared and used for a drug release experiment whereby 10 mL of PBS (10 mM pH 7.4) was added to the films and at specified time points, 100 μL of solution was collected and added to 400 μL of water. The absorbance of these solutions was then measured at 420 nm by uv/vis absorbance.

### Example 4: Case Reports of Therapeutic Efficacy

In all cases, therapeutic efficacy studies were carried out with volunteer participants under the supervision of a healthcare practitioner. In all cases, informed consent was obtained. Participants were blinded to the DBC content of the gel samples provided. Participants were offered samples of control cannabinoid gel (no DBC) and asked to apply to the affected area twice daily and as needed for one week. Participants were told to maintain adherence to any prescribed medications while using the cannabinoid gel. Participants were contacted immediately after the one week treatment period for assessment. Following a washout period ranging from 4 to 12 weeks, participants were offered samples of DBC cannabinoid gel and asked to apply in the same manner, twice daily and as needed for one week, while maintaining adherence to any prescribed medications. Participants were contacted immediately after the one week treatment period for assessment. Efficacy was assessed using average daily 10-point visual analogue scale (VAS) pain score prior to beginning treatment and immediately after treatment. Participants were also asked to report all adverse effects experienced, if any, such as skin irritation, skin rash, localized or diffuse discomfort or pain, dizziness, fatigue, impaired concentration, feelings of euphoria or dysphoria, or other psychotropic effects.

### Case Report 1: Osteoarthritis

LM is a 75 year old woman diagnosed with bilateral osteoarthritis (OA) of the knees at age 59 by her family physician. LM's OA has worsened in severity over the past 4 years and she has a scheduled total knee replacement surgery. The OA pain experienced by LM is described as aching, soreness, a swelling feeling, and sometimes burning feeling at the back of the knee and under the knee cap. Every day or two while walking, LM states her knee joint feels like it “slips and grabs”, causing pain & soreness. Pain and soreness can be improved by sitting and while at rest. LM describes the pain as “nagging” and “constant”. Using a 10-point visual analogue scale (VAS), LM describes the average daily pain level as 3. The pain level is usually 4 on days following extended exercise such as hiking. LM takes extended-release acetaminophen 1300 mg to prevent knee soreness prior to exercise such as hiking. LM also takes ibuprofen 200 mg, or uses topical diclofenac (Voltaren®) as needed for post-exercise soreness and pain flare-ups. On average, LM takes two to three doses of medication per week for OA symptom management. LM finds acetaminophen provides little to no pain relief or prevention of pain during or after exercise. LM typically uses topical diclofenac or ibuprofen when pain level reaches 4. Ibuprofen reduces the pain level to 3 and diclofenac has not been effective in reducing pain level below 3.5.

Following treatment with the control cannabinoid gel (no DBC), LM noted that following 3 applications, VAS pain level was reduced from 3 to 2. After completing one week of treatment with the control cannabinoid gel, LM reported the gel to be “somewhat helpful,” and average daily VAS pain score ranged from 2-3. Following a washout period of 12 weeks, LM completed one week of treatment with the DBC cannabinoid gel. LM reported that within 30 minutes of the first application, VAS pain level was reduced from 3 to 0.5. LM reported an average daily VAS pain level of 0.5 and described the pain as “no longer noticeable or minimally noticeable.” LM reported no adverse effects were experienced with either gel.

### Case Report 2: Osteoarthritis

EC is a 78 year old male diagnosed with osteoarthritis affecting the thumbs and wrists by his family physician approximately 15 years ago. EC experiences dull, nearly chronic aching pain as well as frequent sharp “twinges” of pain which occur sporadically approximately 10 times per week. He reports having an average daily VAS pain level of 3 or 4 and notes the sporadic sharp pain causes a VAS pain level of 5 or 6. EC reports using Voltaren® occasionally during pain flare-ups and notes that it is “a bit helpful, but not much”, reducing VAS pain level to about 3.

Following treatment with the control cannabinoid gel (no DBC), EC reported an average daily VAS pain level of 2.5 and noted the frequency of sharp pain events was reduced to 6 occurrences during the week of treatment. Following treatment with the DBC cannabinoid gel, EC reported an average daily VAS pain level of 1 and noted reduced frequency and severity of sharp pain events (3 events occurred during the week of treatment and VAS pain level of each event was no more than 4). No adverse effects were reported.

### Case Report 3: Osteoarthritis

BD is a 46 year old female diagnosed with early-stage carpal-metacarpal osteoarthritis by her family doctor. BD reports aching bilateral pain that varies between dull and throbbing in nature. Her VAS pain level varies from 1 to 5 and BD rates the average daily pain level as 3. BD notes that the pain flares commonly after her work day, as her occupation requires manual work with her hands. BD uses acetaminophen 500 mg combined with ibuprofen 200 mg to manage pain flare ups approximately once or twice per week and reports modest effectiveness in reducing VAS pain level from 5 to 4.

Following treatment with the control cannabinoid gel (no DBC), BD reported an average daily VAS pain level of 2.5 with noticeable benefit of applying the gel during pain flare ups. The gel was used alongside BD's usual regimen of oral acetaminophen and ibuprofen during two different pain flare ups and the VAS pain level was reduced from 5 to 3 on both occasions. Following treatment with the DBC cannabinoid gel, BD reported an average daily VAS pain level of 1.5 and noted only 1 pain flare-up during the treatment period. During the pain flare-up, BD applied the DBC cannabinoid gel and took oral acetaminophen and ibuprofen. BD noted this was effective in reducing the VAS pain level from 5.5 to 2. No adverse effects were reported.

### Case Report 4: Rheumatoid Arthritis

DM is a 73 year old male with diagnosed rheumatoid arthritis primarily affecting the elbows and hands bilaterally. DM experiences daily soreness, aching and stiffness of the affected joints with some mild swelling apparent. Pain and stiffness are typically worst in the morning. His average daily VAS pain level reportedly fluctuates between 3 to 4. DM currently takes methotrexate and hydroxychloroquine as DMARD therapy and is under the care of a rheumatologist. To manage pain, DM takes an average of 4 doses of ibuprofen 400 mg per week, during times when his VAS pain level is 4. DM states that ibuprofen is mildly effective and reduces pain level from 4 to 3. DM has tried Voltaren® previously and reported no noticeable reduction in pain.

Following treatment with the control cannabinoid gel (no DBC), DM noted a modest benefit after two days, and reported an average daily VAS pain level of 3, noting that pain levels of 4 were not experienced during the treatment period. Following a washout period of 5 weeks, DM then completed one week of treatment with the DBC cannabinoid gel. Immediately after treatment, DM noted “quite a lot of relief” and reported an average daily VAS pain level of 1.5 to 2. DM reported no adverse effects were experienced with either gel.

### Case Report 5: Primary Dysmenorrhea

SR is a 36 year old female with primary dysmenorrhea. During menses, SR experiences cramping and nearly constant pain that varies between dull to sharp. SR's VAS pain level varies from 5 to 7 during menses if she does not take medication. SR takes diclofenac/misoprostal (Arthrotec®), an average of 3 doses, to treat menstrual pain when pain level reaches 6 or 7. She notes moderate effectiveness of Arthrotec® and reports reduction of VAS pain level to 4. SR notes that she prefers not to take Arthrotec® because she typically experiences adverse gastrointestinal effects after each dose. She is unable to tolerate other non-steroidal anti-inflammatory (NSAID) medication (significant adverse gastrointestinal effects).

SR used the control cannabinoid gel (no DBC) by applying topically over the lower abdomen twice daily for 5 days, beginning at the onset of menses. After completing the treatment she noted an improved average daily VAS pain level of 4. SR noted that pain did flare to VAS of 6 or 7 on two occasions, for which she required two doses of Arthrotec® to manage. SR reported no adverse effects from the control gel. Following a 12 week washout period, SR used the DBC cannabinoid gel twice daily for 5 days, following the same time course as the control gel. After completing treatment, she noted “quite an improvement” with an average daily VAS pain level of 2.5. She reported pain to be “much less noticeable on most days” and experienced one pain flare up, which required one dose of Arthrotec® to manage. SR reported use of Arthrotec® along with the DBC cannabinoid gel reduced VAS pain level from 6 to 2. SR reported experiencing mild euphoria and sleepiness, however, she noted the effects were not bothersome.

### Case Report 6: Primary Dysmenorrhea

AF is a 38 year old female with primary dysmenorrhea. AF experiences menstrual pain she describes as aching along with painful cramping. AF reports an average VAS pain level of 6. She does not take any medication to manage pain. AF requested to apply the DBC cannabinoid gel only as needed during menses. She was instructed to apply the DBC cannabinoid gel topically to the lower abdomen twice daily or as needed. AF reported two applications of the gel on two subsequent days during pain flare ups. AF noted a VAS pain level of 7 during the first pain flare, which was reduced to 3 following application of the gel (approximately 30 minutes following application). AF reported a VAS pain level of 7 during the second pain flare, which was reduced to 4 following application of the gel. AF reported experiencing mild sleepiness and mild euphoria following each gel application. AF noted these effects to be “pleasant and relaxing” and not bothersome.

### REFERENCES AND DEFINITIONS

Elias, P. M. 1983. Epidermal lipids, barrier function, and desquamation. J Invest Dermatol. 80 Suppl( ):44s-49s.
Wertz, P. W., and D. T. Downing. 1996. The nature of the epidermal barrier: biochemical aspects. Adv. Drug Del. Rev. 18:283-294.
Zhang, X., Jackson, J. K., Burt, H. M. 1996. Development of amphiphilic diblock copolymers as micellar carriers of taxol. Int J Pharmaceutics. 132:195-206.
Zhang, X., Burt, H. M., Van Hoff, D., Dexter, D., Mangold, G., Degen, D., Oktaba, A. M., Hunter, W. L. 1997. An investigation of the antitumour activity and biodistribution of polymeric micellar paclitaxel. Cancer Chemother Pharmacol. 40:81-86.
Letchford, K., and Burt, H. M. 2012. Copolymer Micelles and Nanospheres with Different In Vitro Stability Demonstrate Similar Paclitaxel Pharmacokinetics. Mol Pharmaceutics. 9:248-260.
Stella, B., Baratta, F., Della Pepa, C., Arpicco, S., Gastaldi, D., Dosio, F. 2021. Cannabinoid Formulations and Delivery Systems: Current and Future Options to Treat Pain. Drugs. 81:1512-1557.
Bruni, N., Della Pepa, C., Oliaro-Bosso, S., Pessione, E., Gastaldi, D., Dosio, F. 2018. Cannabinoid Delivery Systems for Pain and Inflammation Treatment. Molecules. 23:2478.
Momekova, D., Ivanov, E., Konstantinov, S., Ublekov, F., Petrov, P. D. 2020. Nanocomposite Cryogel Carriers from 2-Hydroxyethyl Cellulose Network and Cannabidiol-Loaded Polymeric Micelles for Sustained Topical Delivery. Polymers. 12:1172.
Paudel, K. S., Hammell, D. C., Agu, R. U., Valiveti, S., Stinchcomb, A. L. 2010. Drug Development and Industrial Pharmacy. 36:1088-1097.
Gattefosse. Efficient skin delivery: no compromise with Transcutol®. 2015. Accessed online: https://www.outsourcing-pharma.com/Library/Efficient-skin-delivery-no-compromise-with-Transcutol-R

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Terms such as “exemplary” or “exemplified” are used herein to mean “serving as an example, instance, or illustration.” Any implementation described herein as “exemplary” or “exemplified” is accordingly not to be construed as necessarily preferred or advantageous over other implementations, all such implementations being independent embodiments. Unless otherwise stated, numeric ranges are inclusive of the numbers defining the range, and numbers are necessarily approximations to the given decimal. The word “comprising” is used herein as an open-ended term, substantially equivalent to the phrase “including, but not limited to”, and the word “comprises” has a corresponding meaning. As used herein, the singular forms “a”, “an” and “the” include plural referents unless the context clearly dictates otherwise. Thus, for example, reference to “a thing” includes more than one such thing. Citation of references herein is not an admission that such references are prior art to the present invention. Any priority document(s) and all publications, including but not limited to patents and patent applications, cited in this specification, and all documents cited in such documents and publications, are hereby incorporated herein by reference as if each individual publication were specifically and individually indicated to be incorporated by reference herein and as though fully set forth herein. The invention includes all embodiments and variations substantially as hereinbefore described and with reference to the examples and drawings.

“DBC” means diblock copolymer, and embodiments disclosed herein include the following: 60:40 MePEG: PLLA, MW 3333 Da “DBC₃₃₃₃”; 57:43 MePEG:PCL, MW 1320 Da “PCL₅”; 40:60 MePEG:PCL, MW 1890 Da “PCL₁₀”; 63:37 MePEG:PCL MW 7900 Da “PCL₂₅”; and 30:70 MePEG:PCL, MW 12000 Da “PCL₆₁”.

In some embodiments, the invention excludes steps that involve medical or surgical treatment.

1. A topical pharmaceutical formulation comprising or consisting essentially of:
a diblock copolymer (DBC) having a molecular weight of ≤10 kDa, comprised of a hydrophilic block polymer copolymerized with a hydrophobic block polymer, wherein the hydrophilic block polymer and the hydrophobic block polymer are present in the DBC in a weight ratio of from 35:65 to 65:35; and,
a hydrophobic active agent entrained with the DBC as a dispersion in a topical pharmaceutical carrier, wherein the hydrophobic active agent has a solubility in water of ≤100 μg/ml.

2. The topical pharmaceutical formulation of claim 1, wherein the hydrophilic block polymer comprises polyethylene glycol (PEG) or methoxy-PEG (MePEG).

3. The topical pharmaceutical formulation of claim 1 or 2, wherein the hydrophobic block polymer comprises a polylactide (PLLA), poly(lactic-co-glycolic acid) (PLGA), polycaprolactone (PCL), polydodecyl glycol, polypropylene glycol (PPG) or polymethyl methacrylate.

4. The topical pharmaceutical formulation of any one of claims 1-3, wherein the active agent is a cannabinoid.

5. The topical pharmaceutical formulation of claim 4, wherein the cannabinoid is cannabigerol (CBG), cannabichromene (CBC), tetrahydrocannabivarin (THCV), tetrahydrocannabinolic acid (THCA), tetrahydrocannabinol (THC), cannabidiol (CBD), or cannabinol (CBN).

6. The topical pharmaceutical formulation of any one of claims 1-3, wherein the active agent is curcumin, finasteride, docetaxel, indomethacin, diclofenac, ibuprofen, naproxen, celecoxib, diflusinal, etodolac, flurbiprofen, capsaicin, psilocin or resveratrol.

7. The topical pharmaceutical formulation of any one of claims 1-6, wherein the hydrophilic block polymer and the hydrophobic block polymer are present in the DBC in a weight ratio of greater than 35:65, or greater than 40:60, or from 40:60 to 60:40.

8. The topical pharmaceutical formulation of any one of claims 1-6, wherein the DBC is present in an amount of from 0.5-20% w/w or 2-6% w/w; and/or the hydrophobic active agent is present in an amount of from 0.1-15% w/w or 1-12% w/w; and/or, the pharmaceutical carrier is present in an amount of from 55-99% w/w or 75-97% w/w.

9. The topical pharmaceutical formulation of any one of claims 1-8, wherein the hydrophobic active agent has a solubility in water of ≤90, 80, 70, 60, 50, 40, 30, 20, 15, 10, 9, 8, 7, 6, 5, 4, 3, 2, or 1 μg/ml.

10. The topical pharmaceutical formulation of any one of claims 1-9, wherein formulation is a cream, lotion, gel, paste, ointment, emulsion, adhesive patch or tape, or film.

11. A method of formulating a topical pharmaceutical formulation comprising:
providing a diblock copolymer (DBC) having a molecular weight of ≤10 kDa, comprised of a hydrophilic block polymer copolymerized with a hydrophobic block polymer, wherein the hydrophilic block polymer and the hydrophobic block polymer are present in the DBC in a weight ratio of from 35:65 to 65:35;
admixing a hydrophobic active agent with the DBC to entrain the hydrophobic active agent with the DBC; and,
admixing the hydrophobic active agent entrained with the DBC with a topical pharmaceutical carrier, to provide the hydrophobic active agent entrained with the DBC as a dispersion in a topical pharmaceutical carrier, wherein the hydrophobic active agent has a solubility in water of ≤100 μg/ml.

12. The method of claim 11, wherein the hydrophilic block polymer comprises polyethylene glycol (PEG) or Methoxy-PEG (MePEG).

13. The method of claim 11 or 12, wherein the hydrophobic block polymer comprises a polylactide (PLLA), poly(lactic-co-glycolic acid) (PLGA), polycaprolactone (PCL), polydodecyl glycol, polypropylene glycol (PPG) or polymethyl methacrylate.

14. The method of any one of claims 11-13, wherein the active agent is a cannabinoid.

15. The method of claim 14, wherein the cannabinoid is cannabigerol (CBG), cannabichromene (CBC), tetrahydrocannabivarin (THCV), tetrahydrocannabinolic acid (THCA), tetrahydrocannabinol (THC), cannabidiol (CBD), or cannabinol (CBN).

16. The method of any one of claims 11-13, wherein the active agent is curcumin, finasteride, docetaxel, indomethacin, diclofenac, ibuprofen, naproxen, celecoxib, diflusinal, etodolac, flurbiprofen, capsaicin, psilocin or resveratrol.

17. The method of any one of claims 11-16, wherein the hydrophilic block polymer and the hydrophobic block polymer are present in the DBC in a weight ratio of greater than 35:65, or greater than 40:60, or from 40:60 to 60:40.

18. The method of any one of claims 11-17, wherein the DBC is provided in an amount of from 2-6% w/w; and/or the hydrophobic active agent is provided in an amount of from 1-12% w/w; and/or, the pharmaceutical carrier is provided in an amount of from 75-97% w/w.

19. The method of any one of claims 11-18, wherein the hydrophobic active agent has a solubility in water of ≤90, 80, 70, 60, 50, 40, 30, 20, 15, 10, 9, 8, 7, 6, 5, 4, 3, 2, or 1 μg/ml.

20. The method of any one of claims 11-19, wherein formulation is a cream, lotion, gel, paste, ointment, emulsion, adhesive patch or tape, or film.
